(19) United States
(12) Patent Application Publication
(10) Pub. No.: US 2007/0254290 A1
(43) Pub. Date: Nov. 1, 2007

(54) Biomarkers for diagnosis of crescentic glomerulonephritis

(75) Inventors: Chung-Yang Yen, Taipei City (TW); Ann Chen, Taipei City (TW); Ching-Len Liao, Taipei-City (TW); Jenn-Han Chen, Taipei-City (TW); Wen-Liang Chang, Taipei City (TW); Chen-Wen Yao, Taipei City (TW); Kuo-Yuan Hwa, Taipei City (TW)

Correspondence Address: BIRCH STEWART KOLASCH & BIRCH, PO BOX 747, FALLS CHURCH, VA, 22040-0747, US

(73) Assignee: NATIONAL DEFENSE MEDICAL CENTER

(21) Appl. No.: 11/594,153

(22) Filed: Nov. 8, 2006

(30) Foreign Application Priority Data
Jan. 20, 2006 (TW) 095102192

(51) Int. Cl. C12Q 1/68

(52) U.S. Cl. 435/6

(57) ABSTRACT

This invention discloses a method for diagnosing Crescentic glomerulonephritis (CRGN) using at least a sample from a patient. The method is to find a least one biomarker for diagnosing Crescentic glomerulonephritis. The genetic performance of Sparc, Lcn2 and/or Spp1 can be used as biomarkers.

### BACKGROUND OF THE INVENTION

1. Field of the invention

The present invention relates generally to biomarkers, and more particularly, to biomarkers for diagnosis of crescentic glomerulonephritis.

2. Description of the prior art

Crescentic glomerulonephritis is a syndrome associated with severe glomerular injury, especially with the presence of crescent-shaped structure in the glomerulus. Furthermore, it belongs to an immune disease.

The nephron is the most basic functional unit of the kidney; each nephron is capable of producing urine and it is comprised of a glomerulus, Bowman's capsule, and renal tubule. Furthermore, the glomerulus is a capillary tuft located in the Bowman's capsule. Because these tiny capillaries in the glomerulus are the smallest blood vessels in the kidney, the blood flow is very slow therein, and the molecules in the blood easily deposit on the wall of the tiny capillaries.

The glomerulus is the main filter of the nephron. Water and small molecules in the blood pass through the glomerulus to the renal tubule; furthermore, these molecules are filtered through the basement membrane, formed from both the glomerulus and the Bowman's capsule, to the tubule. The filtered solution will be absorbed and re-absorbed before reaching the pelvis, finally being converted into urine. The pores on the basement membrane have different sizes; only small molecules can pass through the pores, and large molecules will be left outside the basement membrane. However, some of the large molecules may get stuck at the pores and cause severe inflammation.

There are many different kinds of glomerulonephritis, and any thing that chronically stimulates the immune system can cause this kind of kidney damage. Here are some major possible causes:

1. Foreign substances adhere to the glomerular basement membrane and cause the immune response of antibodies against the foreign substances.

2. Foreign substances combine with antibodies to form immune complexes. The immune complexes then deposit itself on the wall of the tiny capillaries of the glomerulus and damage the kidney tissues. Moreover, the immune complexes can be deposited on different locations of the glomerulus to damage different tissues.

3. The immune system creates autoantibodies, which are antibodies or immunoglobulins that attack the kidney cells itself; this is also considered as autoimmune response.

4. Protein structures of the body are altered and induce an autoimmune response because antibodies falsely identify the altered protein structures as foreign substances and start attacking them. Furthermore, the autoantibodies combine with the protein to form immune complexes and deposit itself on the basement membrane of the glomerulus.

Crescentic glomerulonephritis is a kind of glomerulonephritis. One character of the crescentic glomerulonephritis is the proliferation of the epithelial cells on the walls of the Bowman's capsule and the formation of crescent caused by the cell proliferation. As the crescentic glomerulonephritis progresses, crescent structures are formed in most of the glomeruli; furthermore, these crescent structures press on the mass of tiny capillaries of the glomeruli and block the proximal tubules. As a result, the blood flow and filtering rate of the glomeruli are reduced, and the function of the kidney declines.

Crescentic glomerulonephritis belongs to immune complex-mediated disease. Beside the epithelial cells of the Bowman's capsule, the crescent structure further includes monocytes and lymphocytes. Because the crescentic glomerulonephritis is caused by immune system disorder, the pathological symptoms are like the delayed-type hypersensitivity (DTH).

Recently, cytokine cDNA array technology has been applied to examine the cytokine-related genes and chemokine-related genes of the peripheral blood mononuclear of patients with systemic lupus erythematosus, and the results are compared with a control group of healthy cells. Results show that mutant tumor-necrosis factor/death receptors and interleukin-1 cytokine family genes are found to be related to systemic lupus erythematosus. Although the mononuclear and the cytokines play key roles in the development of the lupus nephritis, expressions of genes of renal cells should not be neglected.

In preliminary study, we observed there are extensive crescents associated with either collapsed glomerular tufts or early glomerulosclerosis in a murine chronic graft-versus-host disease model. Furthermore, we further applied cDNA microarry technology, real-time PCR (RT-PCR), and Western blot to analysis the expression of genes, so as to understand and screen the specific proteins as biomarkers in related with the disease; these biomarkers can be used for early detection of the disease in the future.

The present invention is based on the methods described above to screen suitable biomarkers for the diagnosis of CRGN.

### SUMMARY OF THE INVENTION

Accordingly, an aspect of the present invention is to provide specific genes and expression levels thereof as biomarkers for diagnosis of crescentic glomerulonephritis (CRGN).

Furthermore, the present invention provides a method for diagnosis of crescentic glomerulonephritis from a sample of a patient. The method is about screening at least one biomarker from the sample to determine if the patient got CRGN.

The present invention discloses three biomarkers: the first biomarker, the second biomarker, and the third biomarker. Furthermore, each of these biomarkers can be used to diagnose the CRGN. The first biomarker is the increased expression amount of Sparc gene in the sample; the second biomarker is the increased expression amount of Lcn2 gene in the sample; and the third biomarker is the increased expression amount of Spp1 gene in the sample.

Additionally, in another preferred embodiment, the first biomarker is the expressed protein encoded by the Sparc gene in the sample; the second biomarker is the expressed protein encoded by the Lcn2 gene in the sample; and the third biomarker is the expressed protein encoded by the Spp1 gene in the sample.

These biomarkers are capable of helping the diagnosis of CRGN at early stage, and to treat the disease as early as possible.

The aspect of the present invention will no doubt become obvious to those of ordinary skill in the art after reading the following detailed description of the preferred embodiment, which is illustrated in the various figures and drawings.

### BRIEF DESCRIPTION OF THE APPENDED DRAWINGS

FIG. 1 is a list of primers used in the present invention.

FIG. 2 shows the proteinuria and renal function of the CRGN in different weeks.

FIG. 3 shows the confirmation of altered gene expression in the renal cortex of the CRGN model.

FIG. 4 shows the gene expression of glomerular cells of the CRGN model and the normal control in different weeks.

FIG. 5 shows the total intensity score of mRNA in different weeks.

FIG. 6 shows the total intensity score of TGF-β1 mRNA.

FIG. 7 shows the detection and levels of Sparc, Lcn2, and Spp1 in urine during the course of glomerular lesion development.

### DETAILED DESCRIPTION OF THE INVENTION

The present invention provides a method of diagnosing crescentic glomerulonephritis (CRGN) in at least one sample of a patient. The method is to screen specific biomarkers related to CRGN from the sample for the evidence of the determine of CRGN.

The biomarkers of the present invention can be specific genes and the expression levels of the genes, such as the protein synthesis levels. Additionally, the present invention provides three different biomarkers: Sparc, Lcn2, and Spp1. Each of the three biomarkers can be applied to diagnose CRGN. Particularly, the sample of the present invention can be easily collected from urine.

In the present invention, Sparc, Lcn2 and Spp1 are demonstrated to be biomarkers for diagnosis of CRGN. The animal model of the present invention is described below.

Animal model

1. Mice used herein as the animal model were induced with the crescentic glomerulonephritis. Age-matched untreated mice were used as normal controls.

2. Renal tissues were snap-frozen or fixed in 10% buffer formalin for routine histopathology evaluation, immunofluorescence, or in situ hybridization. Semiquantitative evaluation is used in the present invention. Forty or more glomeruli were examined on each slide and assigned values of staining intensity from 0 to 3+. The total intensity score was calculated for the three major components, including (i) Bowman's capsule or crescent epithelial cells; (ii) podocytes; and (iii) mesangial cells, in accordance with the following equation for each specimen:

Total intensity score=(% glomeruli intensity negative×0)+(% glomeruli intensity trace intensity×0.5)+(% glomeruli intensity 1+×1)+(% glomeruli intensity 2+×2)+(% glomeruli intensity 3+×3). The values ranged from 0 to a maximum of 300.

3. cDNA microarray is applied to analysis the mRNA expression of the mice. The cDNA microarray used herein contains 15,000 different mouse cDNA clones, and it is provided by Biochip R&D Center, Tri-Service General Hospital, Taipei, Taiwan.

4. Laser microdissection (LMD) was used to obtain the tissue sample after 3, 6 and 9 weeks of the induction.

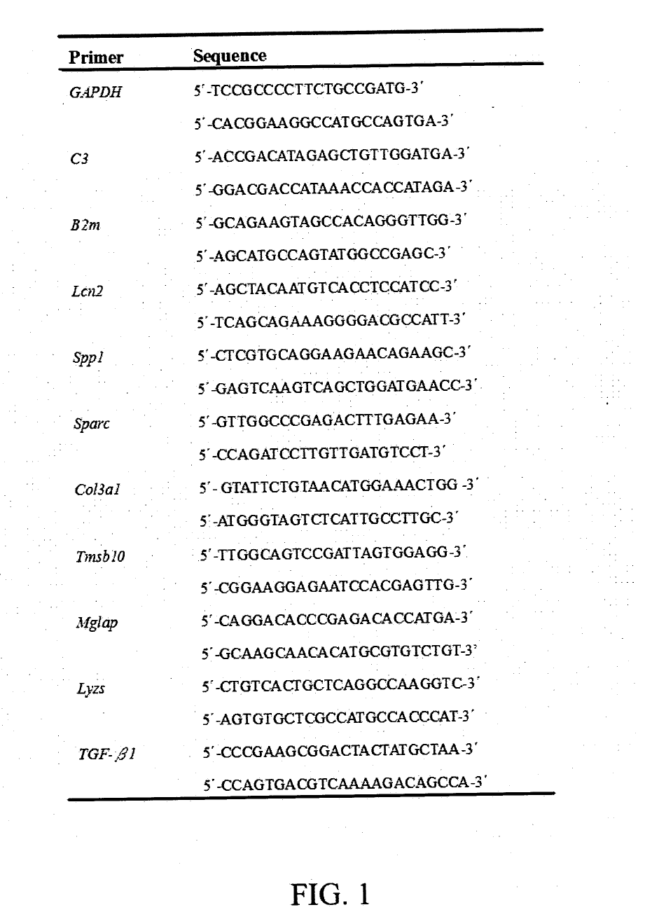

5. With the tissue sample, Real-time PCR (RT-PCR) was used to verify gene expressions detected in the cDNA microarray. As an internal control, glyceraldehyde 3-phosphate dehydrogenase (GAPDH) was added to replace the primers in the reaction. Please refer to FIG. 1 for the primers used herein.

6. Urine samples were prepared for Western Blot Analysis. Moreover, urine creatinine standardized sample was used to examine the Sparc, S100a6, Anxa2, Lcn2 and Spp1 in each of the urine samples.

7. Values were presented as the mean±SE. Furthermore, individual experimental group means of data were compared with controls using the Student t-test. A P-value of <0.05 was considered statistically significant.

Results

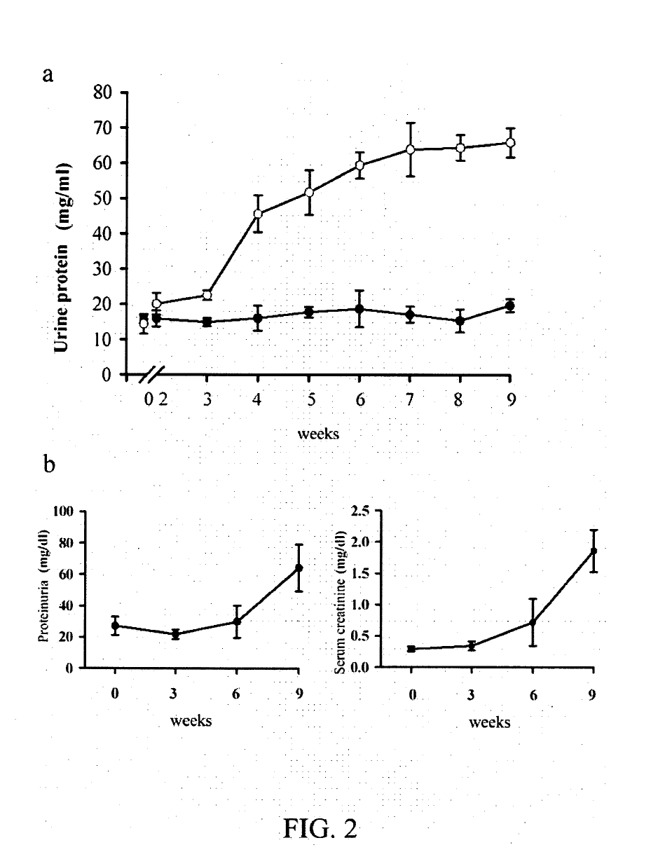

Referring to FIG. 2, FIG. 2 shows the time course studies of urine protein levels and renal function. FIG.2 illustrates that the mice have been induced with the disease in the animal model, and the mice with CRGN are called CRGN mice for short. In FIG. 2, open circle represents the CRGN mice, and the filled circle represents the normal controls. Moreover, each circle was represented as mean value. FIG. 2a shows the proteinuria levels; and FIG. 2b shows the blood urea nitrogen (BUN) levels and the serum creatinine levels of CRGN mice. Moreover, as shown in FIG. 2, there is no significant difference between the CRGN mice and the normal controls at week 3.

As shown in FIG. 2a, the mice developed an increasing proteinuria that was detectable at week 3 after the induction of the CRGN model, and and it progressively increased reaching a plateau by week 6, and remained at this high level to week 9. Furthermore, hematuria was identified in a few of the mice at 6 and 9 weeks, respectively.

As shown in FIG. 2b, elevation of proteinuria and serum creatinine levels were observed. Additionally, progressive impairment of renal function was also observed by elevation of serum creatinine levels. Pathologically, at week 6, the CRGN mice showed thickening of glomerular capillary walls, and formation of the crescent. Furthermore, at week 9, crescent formation in 60-80% of the glomeruli, furthermore, thrombus and sclerosis formed by the blood fibroblast proteins have been found in the crescent structure, and tubulointerstitial inflammation has been discovered.

Additionally, in comparison with the normal controls, result of IF analysis shows IgG and C3 deposition in a granular pattern along the glomerular capillary wall of the CRGN mice. The deposition of IgG and C3 start early at week 3, with greatly increased fluorescence at week 9. Furthermore, IgA and IgM deposition are also identified in a similar pattern, with a much lower fluorescence intensity which represents a much lower amount of both IgA and IgM (data not shown).

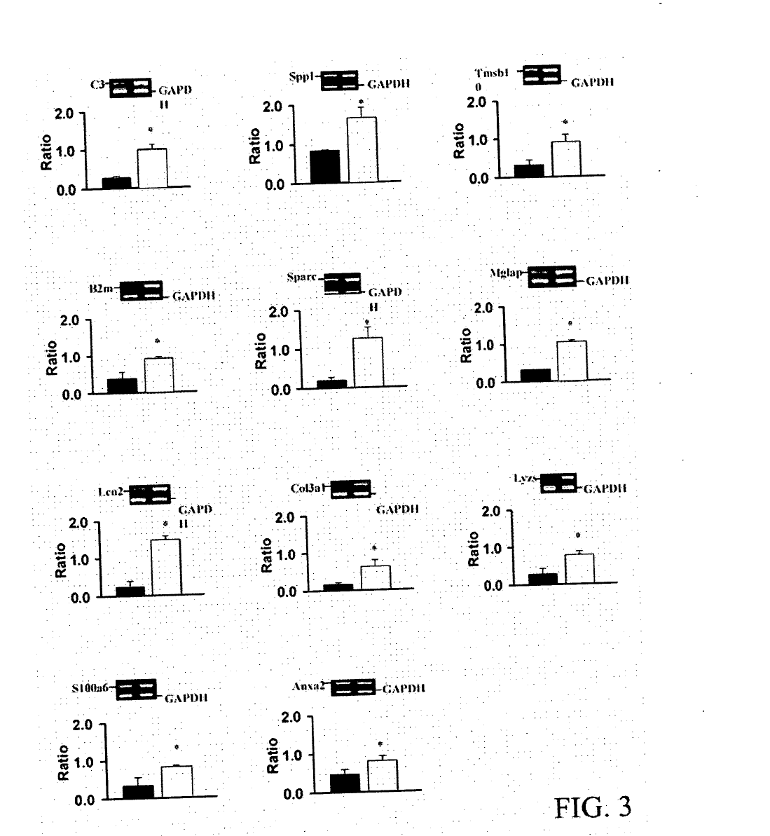

Please refer to FIG. 3, which shows the gene expression in the renal cortex of the CRGN model. This figure represents the RT-PCR results from normal controls and CRGN mice. The test is carried out on the 9th week after the induction. The band density of the RT-PCR product was calculated, and the results are expressed as ratios of the target gene to the internal control GAPDH.

At week 9, abnormal expression of 25 genes was observed in cells from cortical renal tissue of the CRGN mice. Of these genes, 22 of these genes have increased expression, whereas other 3 genes have decreased expression. Further more, 11 of these genes with increased expression including Sparc, Tmsb10, S100a6, Anxa2, Lcn2, Spp1, Col3a1, Mglap, C3, B2m and Lyzs, were confirmed by RT-PCR. The expression levels of these 11 genes were shown in FIG. 3, and each of them is significant higher than normal controls (p<0.05).

As shown in FIG. 3, the expression levels of Sparc, Lcn2, and Spp1 are significantly higher than other genes.

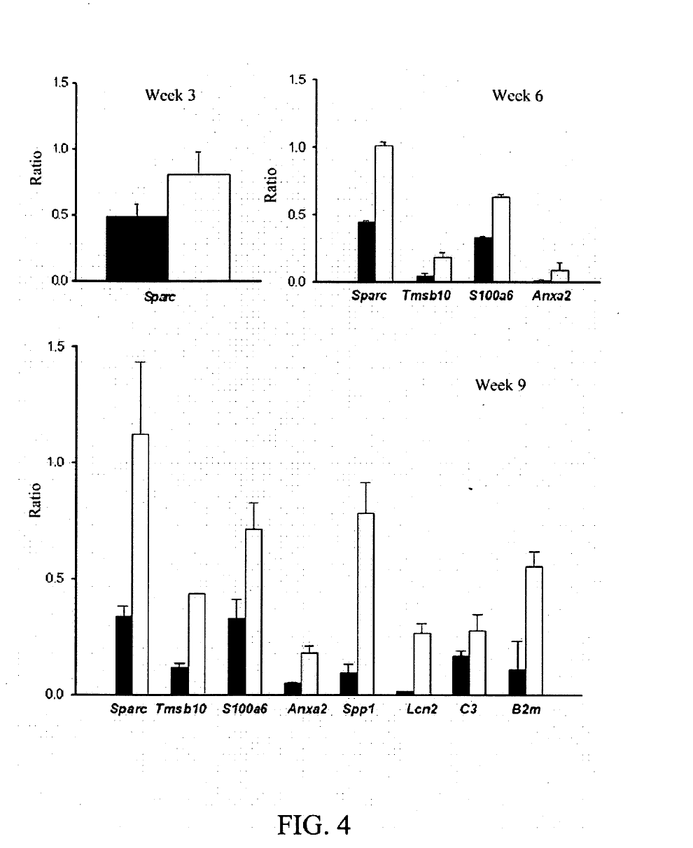

Please refer to FIG. 4, which shows the comparison of the expression levels of genes in the CRGN mice and the normal controls. The gene expression levels were verified by RT-PCR at week 9. Through the certification of RT-PCR, enhanced expression of 8 genes including Sparc, Tmsb10, S100a6, Anxa2, Lcn2, Spp1, C3, and B2m was identified. Between these 8 genes, expression of Sparc was upregulated very early at week 3, followed by that of Tmsb10, S100a6 and Anxa2 at week 6, and then that of Lcn2, Spp1, C3 and B2m at week 9. Compared with the normal controls, there was a significant increase in the expression (mRNA level) of almost all the genes in the CRGN mice at week 9.

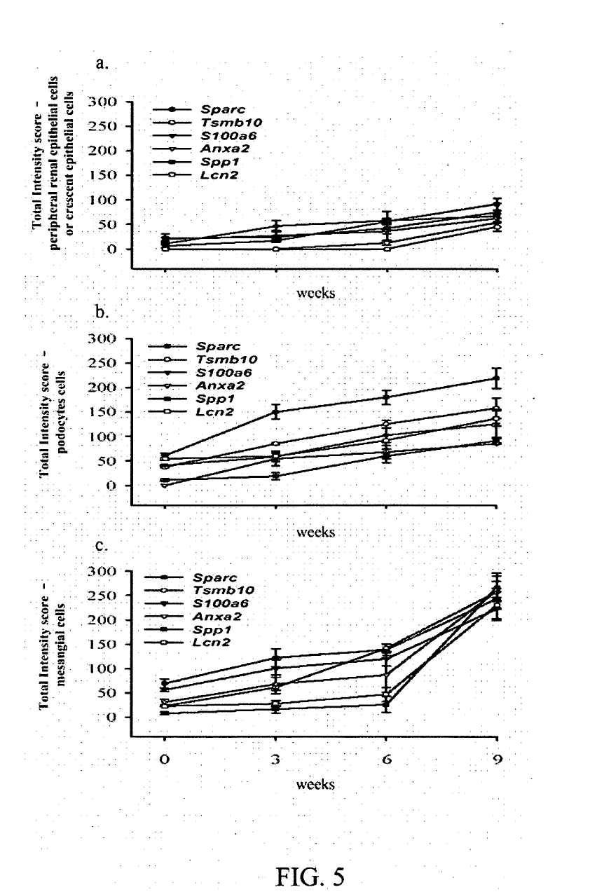

Please refer to FIG. S, which shows the scoring of mRNA expression by In situ hybridization (ISH) at the third, sixth and ninth week. In FIG. 5, each point represents the mean±SE for six mice per group. Furthermore, the score is given for the three major cell types including the renal epithelial cell or the crescent epithelial cells (5a), the podocytes (5b), and the mesangial cells (5c). The total score was calculated as described above.

ISH analysis shows a time-dependent increase in mRNA expression for Sparc, Tmsb10, S100a6, Anxa2, Lcn2, and Spp1 in the CRGN model. At week 3, the expression of Sparc is significantly increased in the podocytes before the formation of crescents. Furthermore, at week 9, when crescent formation was present, the experimental mice showed the most extensive and intensive MRNA expression of all these genes.

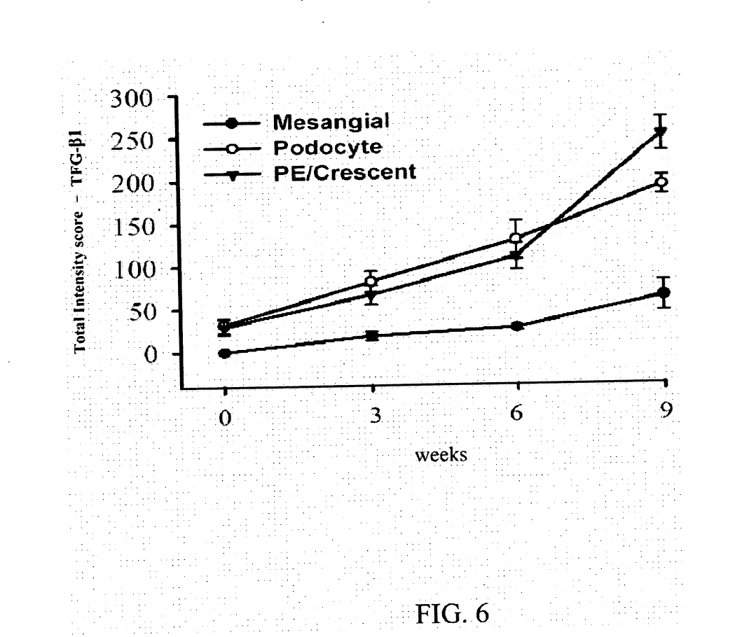

Referring to FIG. 6, FIG. 6 shows the scoring of mRNA expression of transforming growth factor-β1 by ISH. Also, in FIG. 6, each point represents the mean±SE for six mice per group. Enhanced expression of TGF-β1 and Sparc was observed at week 9, when the crescent formation was present and fully developed.

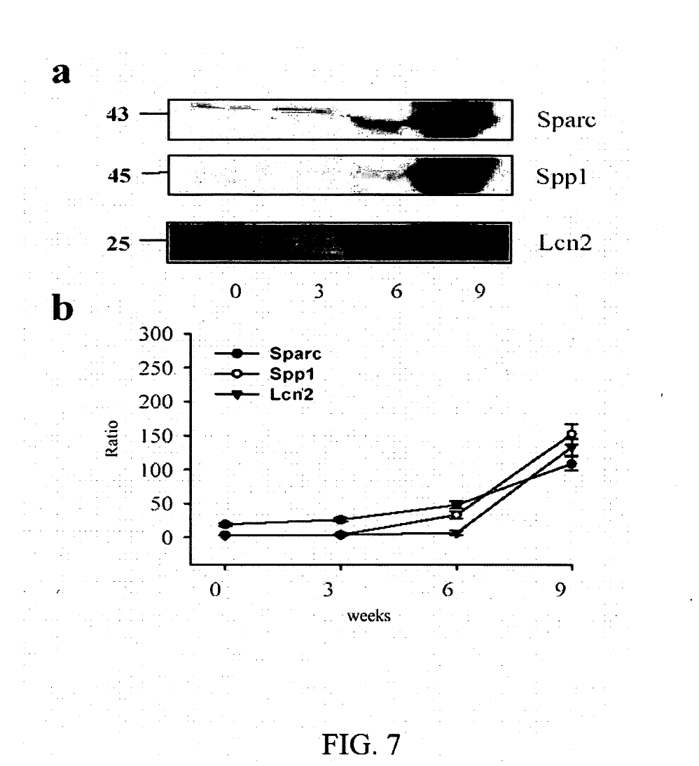

Please refer to FIG. 7, which shows detection and levels of Sparc, Lcn2 and Spp1 in urine during the course of glomerular lesion development. In addition, FIG. 7a shows western blots of the urine samples (10 μl per lane), probed with antibodies against Sparc, Lcn2, and Spp1. Molecular weight markers are shown on the left. FIG. 7b shows semi-quantitative analysis as the ratio of the density to urinary creatinine (in mg/dl). Each point represents the mean±SE for six mice per group.

At week 9, since the expression levels of Sparc, Lcn2, and Spp1 were increased significantly, the proteins encoded by these genes can be detected at high level in urine. On the contrary, there was only trace amount of Sparc, and no detectable amounts of Lcn2 and Spp1 in urine for the normal controls.

Moreover, in the Western blot analysis, there were only traces amounts of S100a6 and Anxa2 in urine for both the CRGN sample and the normal controls. Consequently, both of S100a6 and Anxa2 cannot be used to diagnosis the CRGN.

Through the utilization of microarray technology in the experiments for the animal model as described above, enhanced expression of 8 genes including Sparc, Tmsb10, S100a6, Anxa2, Lcn2, Spp1, C3, and B2m was identified.

Furthermore, the protein levels of Sparc, Lcn2 and Spp1 in urine were significantly elevated in the CRGN in a time-course manner. More importantly, by observing the expression levels of these three proteins, we can evaluate the development of crescent formation.

During the progress of CRGN, both the podocytes and the glomerular crescents expressed Sparc. Because of the early expression of Sparc in the podocytes and then in the glomerular crescents, Sparc is thought to play an important role in the CRGN progress. Moreover, the metabolic pathway of Sparc may be a TGF-β1-dependent pathway.

TGF-β1 is a matricellular protein, it can regulate the interaction between cell and extracellular matrix proteins, so as to inhibit cell proliferation and adhesion of a number of cell types, such as endothelial cells, fibroblasts, and mooth muscle cells. TGF-β1 was upregulated and distributed in the glomerulus in a pattern comparable with Sparc. Additionally, enhanced expression of Sparc can also be observed in renal tissue for passive Heymann nephritis, anti-Thy-1 nephritis, and Diabetes nephritis. In the experiments of the present invention, we found that Sparc can regulate the progressive formation of sclerosis, through the TGF-β1-dependent pathway.

Lcn2 and Spp1, organ failure and inflammation-related genes, were overexpressed in the CRGN model of the present invention. Lcn2 belongs to Lcn family, and its encoded protein, a secreted protein, was markedly expressed in the proximal tubules of early ischaemic mouse kidney.

Spp1 is a cell adhesion and migration molecule, and it acts through binding to ligand, such as integrin v, integrin 3, and extracellular matrix proteins, e.g. type I collagen, fibronectin, and CD44.

In the experiments of the invention, it is the first demonstration of the expression of Lcn2 in glomerular crescents. In comparison with Sparc, although both Spp1 and Lcn2 highly expressed in the late stage of the CRGN model, the expression of both proteins might reflect the influence of influx of mediators of macrophages and lymphocytes on the epithelial cells in the crescent. These findings from the present invention suggest that upregulated expression of Sparc, Tmsb10, S100a6, Anxa2, Lcn2, and Spp1 is closely associated with the development of glomerular lesions, especially crescent formation. During the early stage and process of CRGN, Sparc may play a critical role through the metabolic pathway of TGF-β1. In conclusion, the expression levels of Sparc, Lcn2, and Spp1 are higher than the other genes (as shown in FIG. 3).

Specific expression properties of Sparc, Lcn2, and Spp1 are described below:

Sparc: Sparc was detected early on week 3 before the onset of proteinuria and continued to increase throughout the course of the disease. The early expression of Sparc was limited to the podocytes. However, as the disease progressed, both the podocytes and the glomerular crescents expressed Sparc.

Lcn2: the expression level of Lcn2 in the epithelial cells of renal peripheral was increased significantly on the 9th week after the induction.

Spp1: the expression level of Spp1 in the epithelial cells of renal peripheral was increased significantly on the 9th week after the induction of the disease.

In conclusion, according to the present invention, the expression levels of Sparc, Lcn2, and/or Spp1 are valuable to be biomarkers for diagnosing the crescentic glomerulonephritis. More importantly, detection of Sparc, Lcn2, and/or Spp1 in urine can serve as important biomarkers for diagnosis of CRGN.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A method of diagnosing crescentic glomerulonephritis in at least one sample of a patient, the method being to obtain at least one biomarker from the at least one sample to be an evidence of the crescentic glomerulonephritis, the at least one biomarker comprising one selected from the group consisting of a first biomarker, a second biomarker, and a third biomarker; wherein the first biomarker is the increased expression level of Sparc gene in the sample, the second biomarker is the increased expression level of Lcn2 gene in the sample, and the third biomarker is the increased expression level of Spp1 gene in the sample.

2. The method of claim 1, wherein the at least one sample comprises a podocyte, and the Sparc gene is expressed in the podocyte.

3. The method of claim 1, wherein the at least one sample comprises a glomerular crescents tissue, and the Sparc gene is expressed in the glomerular crescents tissue.

4. The method of claim 1, wherein the Sparc gene is expressed through the synthesis of Sparc protein.

5. The method of claim 1, wherein the at least one sample comprises a renal epithelial cell, and the Lcn2 gene is expressed in said renal peripheral epithelial cell.

6. The method of claim 1, wherein the Lcn2 gene is expressed through the synthesis of Lcn2 protein.

7. The method of claim 1, wherein the at least one sample comprises a renal epithelial cell, and the Spp1 gene is expressed in said renal peripheral epithelial cell.

8. The method of claim 1, wherein the Spp1 gene is expressed through the synthesis of Spp1 protein.


SEQUENCE LISTING (22 sequences)

SEQ ID NO: 1 - DNA, 19 bp, Artificial Sequence
  Description of Artificial Sequence  Synthetic oligonucleotide primer
tccgccccttctgccgatg

SEQ ID NO: 2 - DNA, 21 bp, Artificial Sequence
  Description of Artificial Sequence  Synthetic oligonucleotide primer
cacggaaggccatgccagtga

SEQ ID NO: 3 - DNA, 23 bp, Artificial Sequence
  Description of Artificial Sequence  Synthetic oligonucleotide primer
accgacatagagctgttggatga

SEQ ID NO: 4 - DNA, 23 bp, Artificial Sequence
  Description of Artificial Sequence  Synthetic oligonucleotide primer
ggacgaccataaaccaccataga

SEQ ID NO: 5 - DNA, 22 bp, Artificial Sequence
  Description of Artificial Sequence  Synthetic oligonucleotide primer
gcagaagtagccacagggttgg

SEQ ID NO: 6 - DNA, 21 bp, Artificial Sequence
  Description of Artificial Sequence  Synthetic oligonucleotide primer
agcatgccagtatggccgagc

SEQ ID NO: 7 - DNA, 22 bp, Artificial Sequence
  Description of Artificial Sequence  Synthetic oligonucleotide primer
agctacaatgtcacctccatcc

SEQ ID NO: 8 - DNA, 22 bp, Artificial Sequence
  Description of Artificial Sequence  Synthetic oligonucleotide primer
tcagcagaaaggggacgccatt

SEQ ID NO: 9 - DNA, 22 bp, Artificial Sequence
  Description of Artificial Sequence  Synthetic oligonucleotide primer
ctcgtgcaggaagaacagaagc

SEQ ID NO: 10 - DNA, 23 bp, Artificial Sequence
  Description of Artificial Sequence  Synthetic oligonucleotide primer
gagtcaagtcagctggatgaacc

SEQ ID NO: 11 - DNA, 21 bp, Artificial Sequence
  Description of Artificial Sequence  Synthetic oligonucleotide primer
gttggcccgagactttgagaa

SEQ ID NO: 12 - DNA, 21 bp, Artificial Sequence
  Description of Artificial Sequence  Synthetic oligonucleotide primer
ccagatccttgttgatgtcct

SEQ ID NO: 13 - DNA, 23 bp, Artificial Sequence
  Description of Artificial Sequence  Synthetic oligonucleotide primer
gtattctgtaacatggaaactgg

SEQ ID NO: 14 - DNA, 22 bp, Artificial Sequence
  Description of Artificial Sequence  Synthetic oligonucleotide primer
atgggtagtctcattgccttgc

SEQ ID NO: 15 - DNA, 22 bp, Artificial Sequence
  Description of Artificial Sequence  Synthetic oligonucleotide primer
ttggcagtccgattagtggagg

SEQ ID NO: 16 - DNA, 22 bp, Artificial Sequence
  Description of Artificial Sequence  Synthetic oligonucleotide primer
cggaaggagaatccacgagttg

SEQ ID NO: 17 - DNA, 22 bp, Artificial Sequence
  Description of Artificial Sequence  Synthetic oligonucleotide primer
caggacacccgagacaccatga

SEQ ID NO: 18 - DNA, 23 bp, Artificial Sequence
  Description of Artificial Sequence  Synthetic oligonucleotide primer
gcaagcaacacatgcgtgtctgt

SEQ ID NO: 19 - DNA, 23 bp, Artificial Sequence
  Description of Artificial Sequence  Synthetic oligonucleotide primer
ctgtcactgctcaggccaaggtc

SEQ ID NO: 20 - DNA, 23 bp, Artificial Sequence
  Description of Artificial Sequence  Synthetic oligonucleotide primer
agtgtgctcgccatgccacccat

SEQ ID NO: 21 - DNA, 23 bp, Artificial Sequence
  Description of Artificial Sequence  Synthetic oligonucleotide primer
cccgaagcggactactatgctaa

SEQ ID NO: 22 - DNA, 23 bp, Artificial Sequence
  Description of Artificial Sequence  Synthetic oligonucleotide primer
ccagtgacgtcaaaagacagcca